(19) United States
(12) Patent Application Publication
(10) Pub. No.: US 2007/0015927 A1
(43) Pub. Date: Jan. 18, 2007

(54) New phosphoramidite compounds

(76) Inventors: Byeang Hyean Kim, Kyungsangbuk-do (KR); Su Jeong Kim, Kyungsangbuk-do (KR); Eun-Kyoung Bang, Kyungsangbuk-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/541,769

(22) PCT Filed: Apr. 28, 2003
(86) PCT No.: PCT/KR03/00857

§ 371 Date: May 1, 2006

(30) Foreign Application Priority Data
Jan. 9, 2003 (KR) 10-2003-0001392
Apr. 22, 2003 (KR) 10-2003-0025434

(51) Int. Cl. C07J 51/00; C07F 9/02

(52) U.S. Cl. 552/506; 558/168

(57) ABSTRACT

Novel phosphoramidites having various functional groups can be advantageously used as new building blocks to synthesize various oligodeoxyribonucleotides which are useful for the development of a highly efficient diagnostic agent and the synthesis of new nano structural oligodeoxyribonucleotides.

### FIELD OF THE INVENTION

The present invention relates to a phosphoramidite compound which can be advantageously used to synthesize DNA variants.

### BACKGROUND OF THE INVENTION

There have been attempts to develop a drug based on a DNA synthesized by a chemical method (Agrawal, S., Synthesis and Properties, Humana Press: Totowa, Chapter 1-4, (1993) and Kool, E. T., Chem. Rev., 97; 1473 (1997)). Also, various efforts have been made to develop modified DNAs having interesting structural features (Newcome, G. R., et al., Dendritic Molecules: Concepts, Synthesis, Perspectives, VCH Publishers, New York, 116 (1996); Shchepinov, M. S. et al., Nucleic Acids Res., 25, 4447-4454 (1997); Shchepinov, M. S. et al., Nucleic Acids Res., 27, 3035-3041 (1999); and Winfree, E. et al., Nature, 394, 539-544 (1998)).

In this line, branched DNAs (bDNAs) composed of several oligodeoxyribonucleotide (ODN) strands have been synthesized and studied in order to elucidate the structural and biological characteristics of wild-type bDNAs of interest (Hudson, R. H.; Uddin, A. H.; Damha, M. J., J. Am. Chem. Soc., 117, 12470-12477 (1995); and Collins, M. L. et al., Nucleic Acids Research, Vol. 25, No. 15, 2979-2984 (1997); and Horn, T, et al., Nucleic Acids Research, Vol. 25, No. 23, 4835-4849 (1997)). Such branched DNAs are sometimes used to synthesis a hyperbranched polymer or dendrimer. The dendrimer may be modified by introducing various functional groups at the branch ends thereof so that it can attain a specific biological function (Newkome, G. R. et al., Chem. Rev., 99: 1689-1746 (1999)).

Phosphoramidite compounds coupled with adenosine (A), guanosine (G), cytidin (C) or thymidin (T), have been used to synthesize various wild type DNAs using a DNA synthesizer and such phosphoramidite derivatives can be easily inserted into DNA.

Accordingly, the present inventors have endeavored to develop a new functional phosphoramidite compound, which can be usefully used in synthesizing various oligodeocyribonucleotides (ODNs).

### SUMMARY OF THE INVENTION

Accordingly, it is an object of the present invention to provide a novel phosphoramidite compound which can be used in the synthesis of a desired oligodeocyribonucleotide.

In accordance with an aspect of the present invention, there is provided a phosphoramidite compound of formula (I), (II), (III), (IV) or (V): 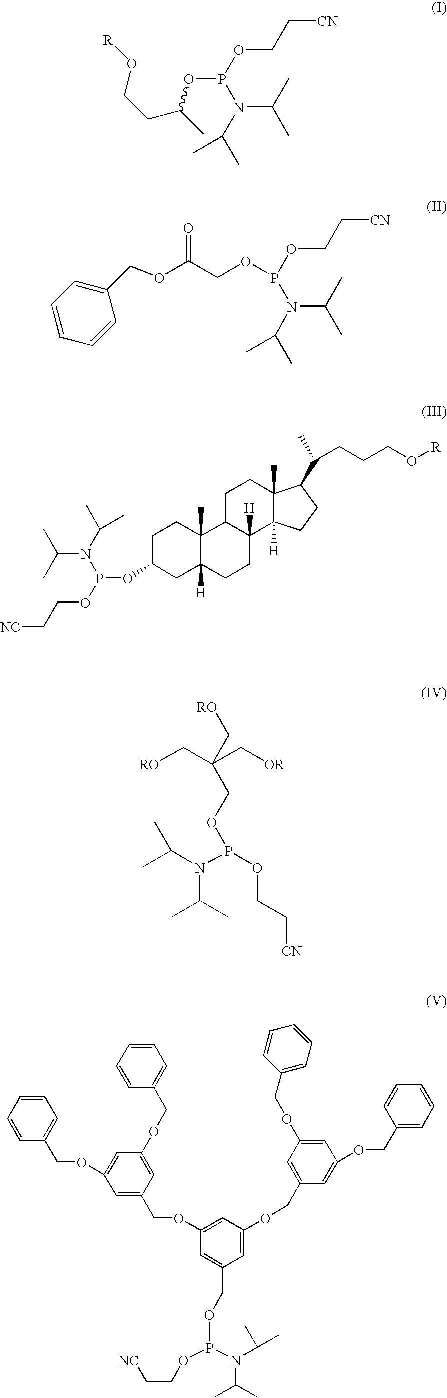
wherein:

R is a dimethoxytrityl(DMTr), levulinyl(Lev) or tert-butyldimethylsilyl (TBDMS) group.

### BRIEF DESCRIPTION OF THE DRAWINGS

The above and other objects and features of the present invention will become apparent from the following description of the invention taken in conjunction with the following accompanying drawings, wherein:

FIG. 1 shows the melting temperatures of synthesized oligomers;

FIGS. 2 and 3 illustrate the CD spectra of synthesized oligomers; and

FIG. 4 are HPLC scans of purified oligomers.

### DETAILED DESCRIPTION OF THE INVENTION

The phosphoramidite compound of the present invention is a compound represented by one of formula (I) to (V): 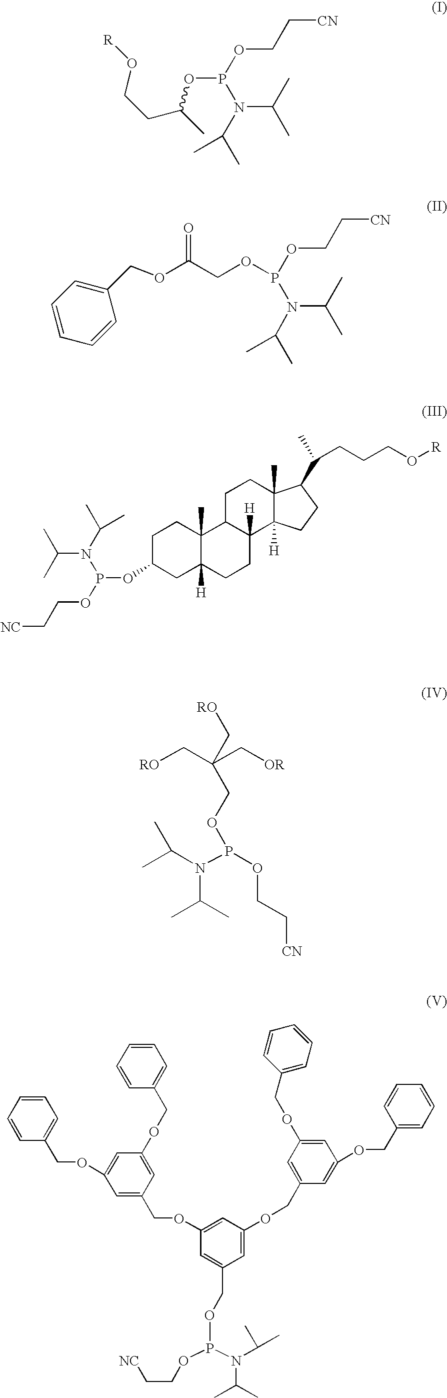
wherein:

R is a dimethoxytrityl(DMTr), levulinyl(Lev) or tert-butyldimethylsilyl (TBDMS) group.

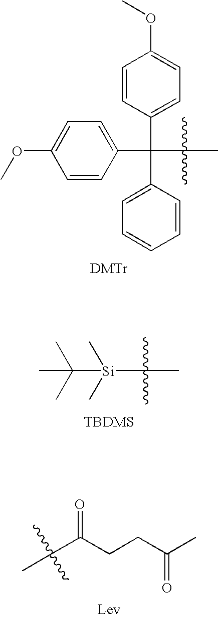

The inventive phosphoramidite compound of formula (I) includes its (S)- and (R)-isomers.

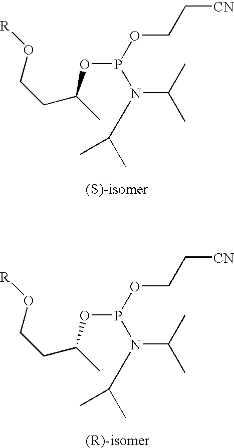

Among the phosphoramidite compounds of the present invention, preferred are:
(S)-(+)-1-O-DMTr-3-O-(2-cyanoethyl)-N,N-diisopropyl-phosphoramidite)-1,3-butanediol;
(R)-(−)-1-O-DMTr-3-O-(2-cyanoethyl)-N,N-diisopropyl-phosphoramidite)-1,3-butanediol;
O-((2-cyanoethyl)-N,N-diisopropyl-phosphoramidite)-benzylglycolate;
O-DMTr-((2-cyanoethyl)-N,N-diisopropyl-phosphoramidite)-lithocholic alcohol;
O-tri-DMTr-((2-cyanoethyl)-N,N-diisopropyl-phosphoramidite)-pentaerithritol;
O-DMTr-O-di-Lev-O-((2-cyanoethyl)-N,N-diisopropyl-phosphoramidite)-pentaerithritol;
O-DMTr-O-Lev-O-TBDMS-((2-cyanoethyl)-N,N-diisopropyl-phosphoramidite)-pentaerithritol; and
dendrimer phosphoramidite compound of formula (V).

The inventive phosphoramidite compound may be prepared by introducing a desired functional group, e.g., 1.3-butanediol, benzyl glycolate or lithocolic acid as described below. A phosphoramidite compound prepared by introducing pentaerithritol or dendrimer, in particular, can be advantageously used in the synthesis of functional branched DNAs (bDNAs).

1) Preparation of an Optically Pure Phosphoramidite Compound of Formula (I) Using (S)-(+)- or (R)-(−)-1,3-butanediol (Scheme 1)

(S)-(+)- or (R)-(−)-phosphoramidite can be prepared by protecting the 1-hydroxyl group of (S)-(+)- or (R)-(−)-1,3-butanediol with a DMtr group to obtain compound 6 or 7 and introducing the phosphoramidite group into the secondary hydroxyl group to obtain the (S)-(+)- or (R)-(−)-phosphoramidite of formula (Ia) or (Ib).

The inventive (S)-(+)- or (R)-(−)-phosphoramidite may be used in linking oligonucleotides as a linker.

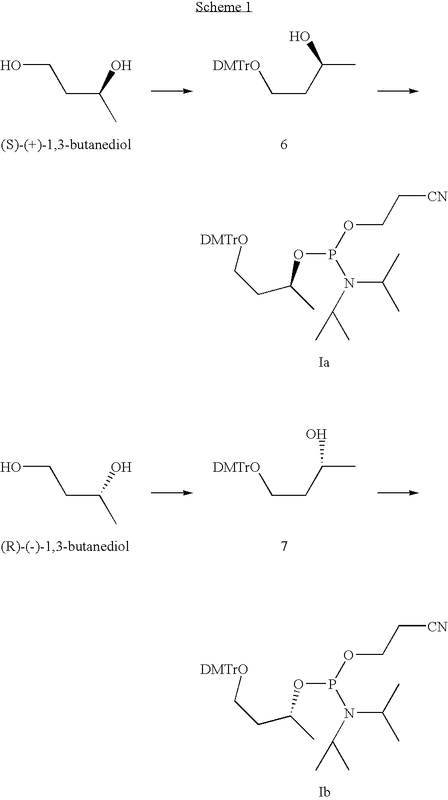

2) Preparation of the Phosphoramidite Compound of Formula (II) Using Benzyl Glycolate (Scheme 2)

The phosphoramidite compound of formula (II) can be prepared by reacting benzyl glycolate and chloro-(2-cyanoethyl)-N,N-diisopropyl-phosphine in THF in the presence of DIPEA (N,N-diisopropylethylamine).

The inventive phosphoramidite compound of formula (II) may be used for introducing an acidic functional group into an oligonucleotide.

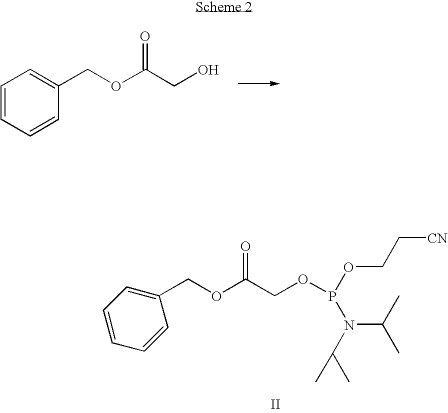

3) Preparation of the Phosphoramidite Compound of Formula (III) Using Lithocholic Acid (Scheme 3)

The phosphoramidite compound of formula (III) can be prepared by reducing the carboxyl group of lithocholic acid to obtain compound 8, protecting the primary hydroxyl group with a DMTr group, and introducing the phosphoramidite group into the secondary hydroxyl group.

The inventive phosphoramidite compound of formula (III) has an enhanced cell permeability due to the hydrophobic lithocholic acid residue and therefore, it may be used in gene therapy. Further, the inventive phosphoramidite compound of formula (III) is expected to be useful in modifying the secondary and third structure of a DNA.

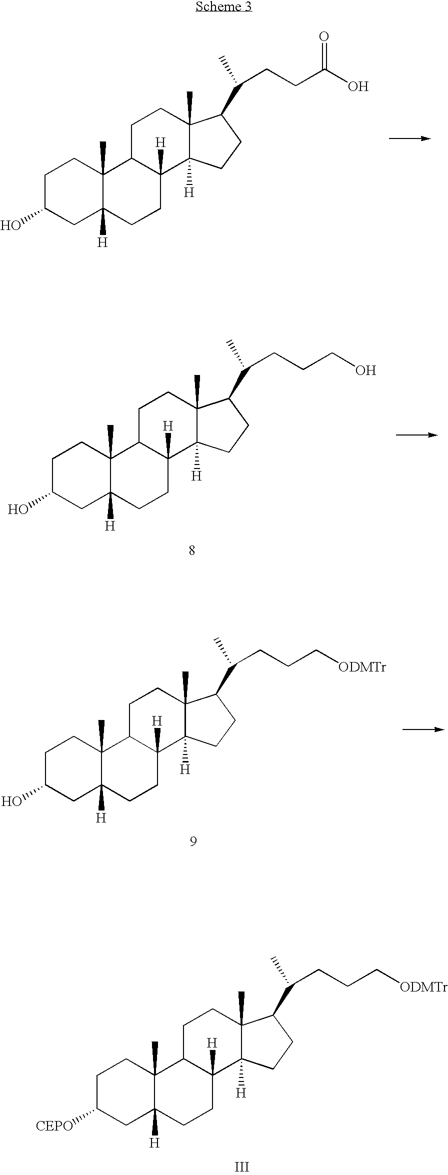

4) Preparation of the Phosphoramidite Compound of Formula (IV) Using Pentaerythritol (Scheme 4)

The phosphoramidite compound of formula (IVa) can be prepared by protecting three of the four hydroxyl groups of pentaerythritol with DMTr groups to obtain compound 10 and introducing the phosphoramidite group into the remaining hydroxyl group. The phosphoramidite compound of formula (IVb) can be prepared by protecting two of the four hydroxyl groups of pentaerythritol with DMTr groups to obtain compound 11, protecting a third hydroxyl group with an Lev group and introducing the phosphoramidite into the remaining hydroxyl group. The phosphoramidite compound of formula (IVc) can be prepared by protecting the hydroxyl group of pentaerythritol with a DMTr group to obtain compound 12, protecting two other hydroxyl groups with Lev groups to obtain compound 14 and introducing the phosphoramidite into the remaining hydroxyl group. Further, the phosphoramidite compound of formula (IVd) can be prepared by introducing a TBDMS (tert-butyldimethylsilyl) group into the compound 15 to obtain compound 12 and introducing the phosphoramidite into the remaining hydroxyl group.

The inventive phosphoramidite compound of formula (IV) may be used in synthesizing dendrimers and bDNAs, especially in synthesizing bDNAs having different base sequences or nano structural oligodeoxyribonucleotides.

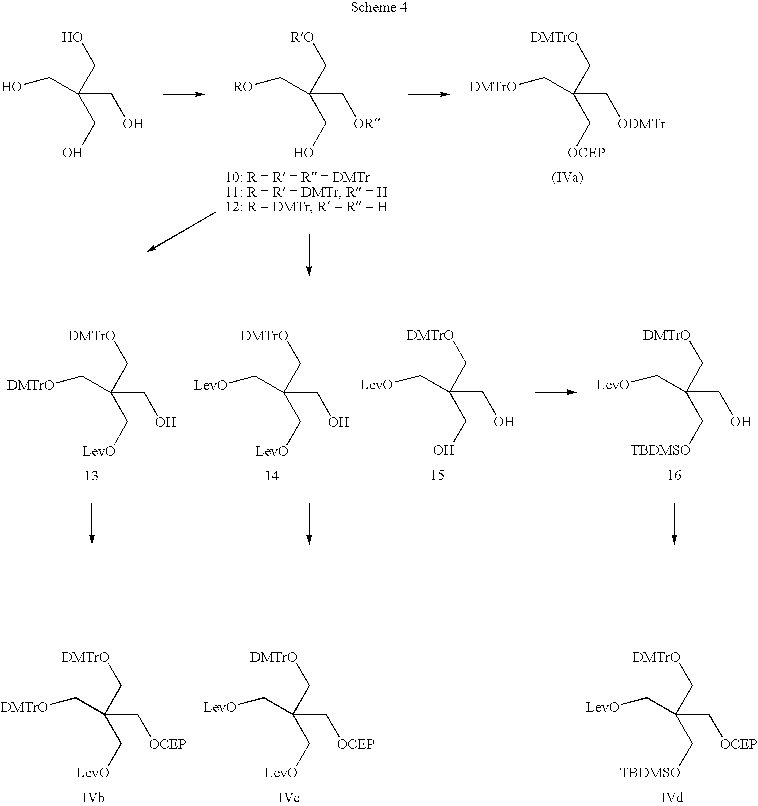

5) Preparation of the Dendrimer Phosphoramidite Compound of Formula (V) Using Dendrimer (Scheme 5)

The dendrimer phosphoramidite compound of formula (V) can be prepared by introducing the phosphoramidite group into the hydroxyl group of dendrimer compound 17

The inventive dendrimer phosphoramidite compound of formula (V) may be used in introducing a dendrimer having desired functional groups into an oligonucleotide.

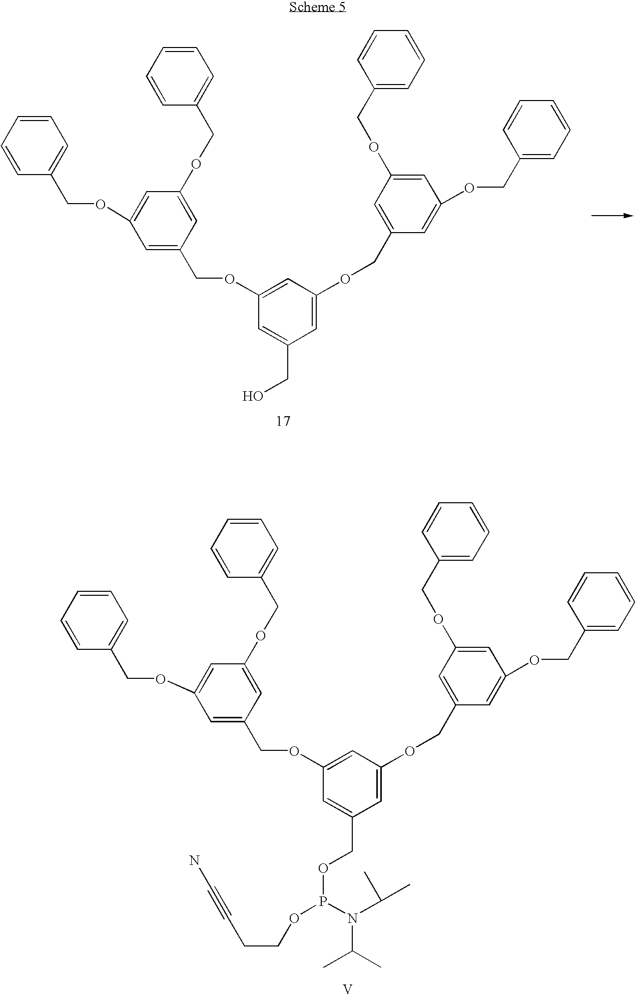

The following examples are intended to further illustrate the present invention without limiting its scope.

### EXAMPLE 1

### Preparation of the Phosphoramidite Compound of Formula (Ia) Using (S)-(+)-1,3-butanediol

(1) Preparation of S-(+)-1-O-(4,4′-dimethoxytrityl)-1,3-butanediol (Compound 6)

S-(+)-1,3-butanediol (96 mg, 1.065 mmol) in 3 ml of pyridine was cooled in an ice-water bath and 4,4′-dimethoxytrityl chloride (430 mg, 1.27 mmol) was added thereto. The resulting mixture was stirred for 6 hours at room temperature. 10 ml of 5% NaHCO₃ was added thereto and the resulting solution was extracted with 15 ml of ethyl acetate. The organic layer was dried over MgSO₄ and evaporated under a reduced pressure. The resulting yellow liquid residue was purified by silica gel column chromatography (eluent—ethyl acetate:hexane=1: 3) to obtain the title compound (401 mg, 1.02 mmol) in a yield of 96%.

R_{f}=0.3 (ethyl acetate:Hexane=1:2); IR (NaCl) ν (cm⁻¹) 3462, 3059, 3034, 2959, 2927, 2848, 2835, 1607, 1508, 1250; ¹H NMR (Acetone-d₆) δ 7.49 (br, 1H), 7.46(br, 1H), 7.36-7.18 (m, 7H), 6.86 (t, 2H, J=2.6 Hz), 6.84 (t, 2H, J=2.6 Hz), 3.93(br, 1H), 3.73(s, 6H), 3.50(br, 1H), 3.28-3.14 (m, 2H), 1.73 (m, 2H), 1.11(d, 3H, J=6.2 Hz); ¹³C-NMR (75.5 MHz, Acetone-d₆) δ 158.1, 145.3, 136.1, 136.0, 129.5, 127.6, 127.2, 126.1, 112.5, 85.4, 64.2, 60.6, 54.2, 39.0, 23.1; MS-FAB (m/z): [M]⁺ calcd for C₂₅H₂₈O₄, 392; found 392.; [α]²¹_{D}=+17.6 (c 1.0, CHCl₃).

(2) Preparation of S-(+)-1-O-DMTr-3-O-((2-cyanoethyl)-N,N-dimethoxytrityl)-1,3-butanediol (Compound Ia)

S-(+)-1-O-(4,4′-dimethoxytrityl)-1,3-butanediol (158 mg, 0.402 mmol) obtained in step 1 was dissolved in 3 ml of THF, DIPEA (140 μl, 0.804 mmol) was added thereto, and then the mixture was stirred for 30 min, followed by adding chloro-(2-cyanoethyl)-N,N-diisopropylamino phosphine (177 μl, 0.80 mmol) dropwise thereto. Then, the white precipitates formed were filtered and dried under a reduced pressure. 20 ml of 5% NaHCO₃ was added thereto and the resulting solution was extracted with ethyl acetate. The organic layer was dried over MgSO₄ and evaporated under a reduced pressure. The resulting yellow liquid residue was purified by silica gel column chlomatography (eluent—ethyl acetate:hexane=1:5) to obtain the title compound (203 mg, 0.34 mmol) as a colorless oil in a yield of 85%.

¹H-NMR (300 MHz, Acetone-d₆) δ 7.47-7.43(2H, m), 7.34-7.25 (5H, m), 7.22-7.16 (1H, m), 6.89-6.80 (4H, m), 4.15 (1H, m), 3.74 (3H, s), 3.73 (3H, s), 3.63-3.51 (3H, m), 3.20-3.16 (2H, m), 2.68 (1H, t, J=6.0 Hz), 2.55 (1H, t, J=6.0 Hz), 1.94-1.73 (3H, m), 1.21-1.11 (12H, m), 1.07 (1.5H, s), 1.05 (1.5H, s); ¹³C-NMR (75.5 MHz, Acetone-d₆) δ 158.1, 145.2, 136.0, 129.6, 129.5, 127.7, 127.6, 127.2, 126.1, 117.7, 117.6, 112.5, 85.4, 68.0, 67.7, 67.4, 67.2, 60.0, 59.8, 59.2, 58.1, 57.8, 57.5, 54.2, 42.4, 42.2, 38.3, 23.7, 23.6, 23.6, 23.5, 23.4, 21.6, 19.5, 19.4; ³¹P-NMR (121 MHz, Acetone-d₆) δ 149.0, 148.3; MS-FAB (m/z): [M+Na]⁺ calcd for C₃₄H₄₅N₂O₅P₁Na₁, 615; found 615.

### EXAMPLE 2

### Preparation of the Phosphoramidite Compound of Formula (Ib) Using (R)-(−)-1,3-butanediol

(1) Preparation of R-(−)-1-O-(4,4′-dimethoxytrityl)-1,3-butanediol (Compound 7)

R-(−)-1,3-butanediol (103 mg, 1.14 mmol) in 3 ml of pyridine was cooled in an ice-water bath, and 4,4′-dimethoxytrityl chloride (460 mg, 1.36 mmol) was added thereto. The resulting mixture was stirred for 6 hours at room temperature. 10 ml of 5% NaHCO₃ was added thereto and the resulting solution was extracted with 15 ml of ethyl acetate. The organic layer was dried over MgSO₄ and evaporated under a reduced pressure. The resulting yellow liquid residue was purified by silica gel column chlomatography (eluent—ethyl acetate:hexane=1:3) to obtain the title compound (437 mg, 1.11 mmol) in a yield of 97%.

R_{f}=0.3 (ethyl acetate:hexane=1:2); IR (NaCl) ν (cm⁻¹) 3462, 3059, 3034, 2960, 2929, 2835, 1607, 1508, 1250; ¹H NMR (Acetone-d₆) δ 7.47 (t, 1H, J=1.7 Hz), 7.45(br, 1H), 7.35-7.20 (m, 7H), 6.87 (t, 2H, J=2.6 Hz), 6.84 (t, 2H, J=2.6 Hz), 3.92(br, 1H), 3.73(s, 6H), 3.47(d, 1H, J=3.7 Hz), 3.25-3.14 (m, 2H), 1.71 (m, 2H), 1.09(d, 3H, J=6.2 Hz); ¹³C-NMR (75.5 MHz, Acetone-d₆) δ 158.1, 145.2, 136.1, 136.0, 129.5, 127.6, 127.2, 126.1, 112.5, 85.4, 64.2, 60.5, 54.1, 38.9, 23.0; MS-FAB (m/z): [M]⁺ calcd for C₂₅H₂₈O₄, 392; found 392.; [α]²¹ _{D}=−9.9 (c 1.0, CHCl₃).

(2) Preparation of R-(−)-1-O-DMTr-3-O-((2-cyanoethyl)-N,N-dimethoxytrityl)-1,3-butanediol

R-(−)-1-O-(4,4′-dimethoxytrityl)-1,3-butanediol (138 mg, 0.315 mmol) obtained in step 1 was dissolved in 2 ml of THF, DIPEA (140 μl, 0.804 mmol) was added thereto and stirred for 30 min, followed by adding chloro-(2-cyanoethyl)-N,N-diisopropylamino phosphine (157 μl, 0.70 mmol) dropwise thereto. Then, white precipitates formed were filtered, dried under a reduced pressure. 10 ml of 5% NaHCO₃ was added thereto and the resulting solution was extracted with 15 ml of CH₂Cl₂. The organic layer was dried over MgSO₄ and evaporated under a reduced pressure. The resulting yellow liquid residue was purified by silica gel column chlomatography (eluent—ethyl acetate:hexane=1:5) to obtain the title compound (108 mg, 0.182 mmol) as a colorless oil in a yield of 52%.

R_{f}=0.45 (ethyl acetate:hexane=1:5) ¹H-NMR (300 MHz, Acetone-d₆) δ 7.47-7.43(2H, m), 7.34-7.25 (5H, m), 7.22-7.16 (1H, m), 6.89-6.80 (4H, m), 4.15 (1H, m), 3.76 (3H, s), 3.75 (3H, s), 3.63-3.51 (3H, m), 3.20-3.16 (2H, m), 2.68 (1H, t, J=6.0 Hz), 2.55 (1H, t, J=6.0 Hz), 1.94-1.73 (3H, m), 1.19-1.10 (12H, m), 1.05 (1.5H, s), 1.03 (1.5H, s); ¹³C-NMR (75.5 MHz, Acetone-d₆) δ 158.1, 145.2, 136.0, 129.5, 129.5, 127.6, 127.5, 127.2, 126.1, 117.6, 112.4, 85.3, 67.9, 67.7, 67.4, 67.2, 66.7, 59.9, 59.8, 58.0, 57.8, 57.5, 54.1, 42.4, 42.2, 38.3, 38.2, 24.8, 23.7, 23.6, 23.5, 23.4, 23.3, 21.6, 19.4, 19.3; ³¹P-NMR (121 MHz, Acetone-d₆) δ 149.0, 148.3; MS-FAB (m/z): [M+Na]⁺ calcd for C₃₄H₄₅N₂O₅P₁Na₁, 615; found 615.

### TEST EXAMPLE 1

The effect exerted by the chiral structural difference of enantiomers on the oligonucleotide structure was examined as follows.

The compounds of formula (Ia) and (Ib) obtained Example 1 and 2 respectively, were each inserted into an oligonucleotide using Expedite Nucleic Acid Synthesis System 8090 and the molecular weight of synthesized ODNs was determined by employing Maldi-Tof mass(Acceleratting voltage: 25000V, Matrix: 3-Hydroxypicolinic acid, Polarity: Positive).

The results are shown in Table 1.

TABLE 1

| | | The molecular weight | |
|---|---|---|---|
| No. | Sequence | calcd | Found |

| | | | |
|---|---|---|---|
| 1 | 5′d-T₆RpT₅ | 3425.3 | 3433.3 |
| 2 | 5′d-T₆SpT₅ | 3425.3 | 3462.3 |
| 3 | 5′d-A₅RpA₆ | 3528.7 | 3534.5 |
| 4 | 5′d-A₅SpA₆ | 3528.7 | 3538.6 |
| 5 | 5′d-T₁₂ | 3577.4 | 3594.0 |
| 6 | 5′d-T₁₁ | 3274.2 | 3277.6 |
| 7 | 5′d-A₁₂ | 3690.2 | 3701.0 |
| 8 | 5′d-A₁₁ | 3377.6 | 3384.1 |
| 9 | 5′d-AACGTTRpAACGTT | 3786.0 | 3794.0 |
| 10 | 5′d-AACGTTSpAACGTT | 3786.1 | 3794.9 |
| 11 | 5′d-AACGTTAAACGTT | 3947.6 | 3950.7 |
| 12 | 5′d-AACGTTTAACGTT | 3938.2 | 3944.7 |
| 13 | 5′d-AACGTTGAACGTT | 3963.2 | 3969.1 |
| 14 | 5′d-AACGTTCAACGTT | 3923.2 | 3925.9 |

Rp: R-(+)-1-O-DMTr-3-O-((2-cyanoethyl)-N,N-dimethoxytrityl)-1,3-butanediol
Sp: S-(+)-1-O-DMTr-3-O-((2-cyanoethyl)-N,N-dimethoxytrityl)-1,3-butanediol

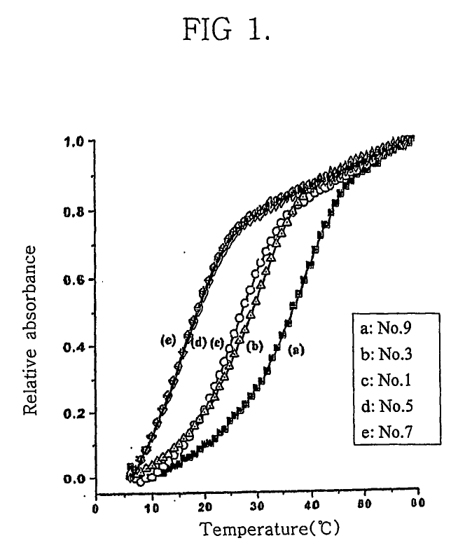

Further, the melting temperatures (Tm) of various duplexes (oligo/oligo) were determined by measuring the changes in the absorbance at 260 nm (cuvette, 1 Cm path length) with increasing temperature at rate of 1.0° C./min using solution in Tris-HCl buffer (10 mM, pH 7.2) containing 100 mM NaCl and 20 mM MgCl₂. The result is shown in Table 1 and FIG. 1. The total concentrations of duplexes 1, 2 and 9 were each adjusted to 4.0 μM, while the total concentrations of duplexes 3 to 8 and 10, to 6.6 μM.

TABLE 2

| | | | | |
|---|---|---|---|---|
| | | | Tm | ΔTm |
| Entry | Duplex | Sequence | (° C.) | (° C.) |

| | | | | |
|---|---|---|---|---|
| 1 | Oligo 1/Oligo 7 | 5′d-T₆RpT₅/ | 26° C. | −12° C. |
| | | 5′d-A₁₂ | | |
| 2 | Oligo 2/Oligo 7 | 5′d-T₆SpT₅/ | 26° C. | −12° C. |
| | | 5′d-A₁₂ | | |
| 3 | Oligo 3/Oligo 5 | 5′d-A₅RpA₆/ | 28° C. | −10° C. |
| | | 5′d-T₁₂ | | |
| 4 | Oligo 4/Oligo 5 | 5′d-A₅SpA₆/ | 27° C. | −11° C. |
| | | 5′d-T₁₂ | | |
| 5 | Oligo 1/Oligo 3 | 5′d-T₆RpT₅/ | 15° C. | −23° C. |
| | | 5′d-A₅RpA₆ | | |
| 6 | Oligo 1/Oligo 4 | 5′d-T₆RpT₅/ | 14° C. | −24° C. |
| | | 5′d-A₅SpA₆ | | |
| 7 | Oligo 2/Oligo 3 | 5′d-T₆SpT₅/ | 15° C. | −23° C. |
| | | 5′d-A₅RpA₆ | | |
| 8 | Oligo 2/Oligo 4 | 5′d-T₆SpT₅/ | 15° C. | −23° C. |
| | | 5′d-A₅SpA₆ | | |
| 9 | Oligo 5/Oligo 7 | 5′d-T₁₂/ | 38° C. | 0° C. |
| | | 5′d-A₁₂ | | |
| 10 | Oligo 6/Oligo 8 | 5′d-T₁₁/ | 35° C. | −3° C |
| | | 5′d-A₁₁ | | |

As shown in Table 2, Tm (° C.) values of the duplex (oligo 1/oligo 7) and duplex (oligo 2/oligo 7) are lower by about 12° C. than that of duplex (oligo 5/oligo 7). Such negative ΔTm value is caused by the substitution of the nucleoside with 1,3-butanediol which has higher flexibility than a sugar moiety and no base that can hydrogen bond to the oligonucleotide. This result suggests that the difference in the chiral structure of a pair of enantiomers does not affect Tm value of the oligonucleotide.

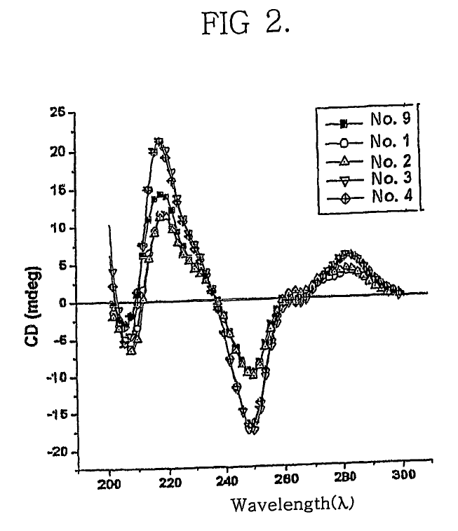

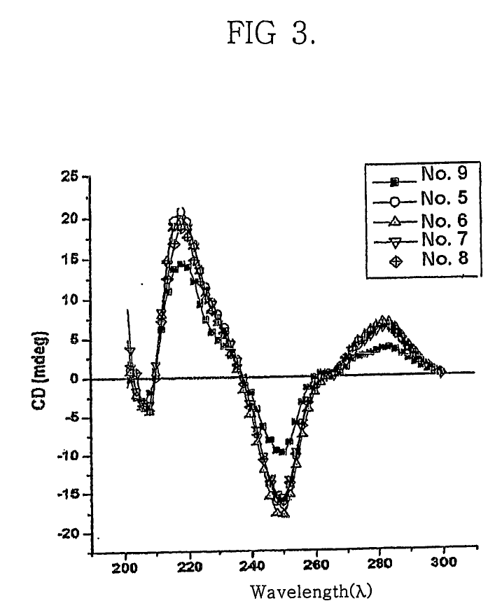

Further, CD (circular dichroism) spectra of the oligomers are shown in FIGS. 2 and 3. As can seen from these results, the double helix structure of the inventive oligomer is similar to that of a wild-type oligomer.

HPLC (high performance liquid chromatography) was also carried out for oligo 1, oligo 2 and a mixture thereof (1:1) under the following conditions.

temperature: room temperature

column: Agilent Eclipse XDB-C18, 4.6×150 mm, 5μ, 80 Å pore size

solvent program: 5% acetonitrile/0.1M triethylammonium acetate (TEAA) (pH 7.0) was eluted for 10 min. Then, the gradient was linearly increased to 50% acetonitrile/0.1M TEAA for 10 min. After 5 minutes, the gradient was linearly recovered to the initial state.

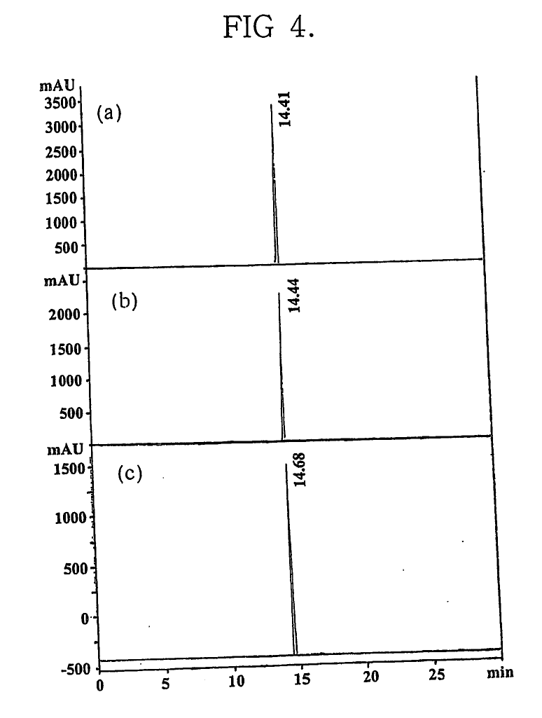

The result in FIG. 4 shows that oligo 1(a), oligo 2(b) and a mixture thereof(c) eluted at the same retention time, suggesting that the structural difference in terms of (S)- and (R)-isomers does not significantly affect the oligonucleotide structure.

### EXAMPLE 3

### Preparation of O-((2-cyanoethyl)-N,N-diisopropyl-phosphoramidite)-benzylglycolate Using benzylglycolate

Benzyl glycolate (100 μl, 0.704 mmol) and DIPEA (480 μl, 2.8 mmol) were added to 7 ml of THF. After stirring for 30 min, chloro-(2-cyanoethyl)-N,N-diisopropyl-phosphine (234 μl, 1.06 mmol) was added dropwise thereto and stirred for 30 min. Then, the bulky white precipitates formed were filtered and dried under a reduced pressure. 25 ml of 5% NaHCO₃ was added thereto and the resulting solution was extracted with 40 ml of CH₂Cl₂. The organic layer was dried over MgSO₄ and evaporated under a reduced pressure. The resulting yellow liquid residue was purified by silica gel column chlomatography (eluent—ethyl acetate:hexane=1:3) to obtain the title compound (167 mg, 0.458 mmol) as a colorless liquid in a yield of 65%.

MS (FAB): m/z: 389.0 [M+Na⁺]; IR (neat): v=3032, 2967, 2932, 1758, 1496, 1455, 1395, 1185, 1098; ¹H-NMR (300 MHz, CDCl₃) δ 7.33 (5H, s), 5.16 (2H, s), 4.28-4.17 (2H, m), 3.91-3.81 (2H, m), 3.64-3.57 (2H, m), 2.63-2.56 (2H, m), 1.77-1.23 (12H, m); ¹³C-NMR (75.5 MHz, CDCl₃) δ 169.8, 169.8, 134.9, 128.1, 128.0, 117.3, 66.3, 60.4, 60.1, 58.6, 58.4, 42.9, 42.7, 24.2, 24.1, 24.0, 19.8, 19.8; ³¹P-NMR (121 MHz, CDCl₃) δ 153.7; HRMS-FAB (m/z): [M+Na]⁺ calcd for C₁₈H₂₇N₂O₄P₁Na₁, 389.1606; found, 368.1603.

### EXAMPLE 4

### Preparation of Phosphoramidite Compound of Formula (III) Using Lithocholic Alcohol

(1) Preparation of Lithocholic Alcohol (Compound 8)

Lithocholic acid (527 mg, 1.40 mmol) was dissolved in 30 ml of THF and cooled to 0° C., followed by adding LAH (lithium aluminum hydride, 247.6 mg, 6.58 mmol) dropwise thereto. After stirring for 4 hours, 250 μl of H₂O and 250 μl of 15% NaOH were sequentially added thereto, followed by adding 750 μl of H₂O thereto. Then, the bulky white precipitates formed were filtered and dried under a reduced pressure to obtain the title compound (486.4 mg, 1.33 mmol) as a white solid in a yield of 95%.

m.p. 96.5-97.8° C.; MS (EI): m/z: 362.3 [M⁺]; IR (neat): v=3205, 2934, 2862, 1446, 1066, 914, 728 cm⁻¹; ¹H-NMR (300 MHz, CDCl₃) δ=3.61-3.57 (3H, m), 1.82-1.01 (28H, m), 0.90 (6H, s), 0.62 (3H, s); ¹³C-NMR (75.5 MHz, CDCl₃) δ 70.3, 62.0, 56.0, 55.7, 42.1, 41.6, 35.9, 35.3, 35.1, 35.0, 34.1, 31.5, 30.0, 29.0, 27.8, 26.8, 26.0, 23.7, 23.0, 20.3, 18.2, 11.6; HRMS-FAB (m/z): [M-OH]⁺ calcd for C₂₄H₄₁O₁, 345.3157; found, 345.20.

(2) Preparation of O-DMTr-Lithocholic Alcohol (Compound 9)

Lithocholic alcohol (455.1 mg, 1.25 mmol) obtained in step 1 and DMAP (68 mg, 0.06 mmol) were dissolved in 10 ml of pyridine, DMTr-Cl (544 mg, 1.63 mmol) was added thereto and stirred for 19 hours at room temperature. 50 ml of 5% NaHCO₃ was added thereto and the resulting solution was extracted with 50 ml of ethyl acetate. The organic layer was dried over MgSO₄ and evaporated under a reduced pressure. The resulting orange colored oil residue was purified by silica gel column chromatography (eluent—ethyl acetate:hexane=1:4) to obtain the title compound (744.8 mg, 1.12 mmol) as a white solid in a yield of 89%.

m.p. 81.2-82.1° C.; MS (FAB): m/z: 664.4 (M⁺); IR (neat): v=3421, 2934, 2863, 1739, 1608, 1582, 1509, 1446, 1250, 1175, 1036, 827 cm⁻¹; ¹H-NMR (300 MHz, CDCl₃) δ=7.45 (d, 2H, J=7.2 Hz), 7.35-7.26 (m, 7H), 6.89-6.80 (dd, 4H, J₁=7.0 Hz, J₂=1.9 Hz), 3.80 (s, 6H), 3.64 (br, 1H), 3.04-2.98 (m, 2H), 1.99-0.89 (m, 34H), 0.63 (s, 3H); ¹³C-NMR (75.5 MHz, CDCl₃): δ=159.0, 148.2, 137.6, 130.7, 129.0, 128.3, 127.2, 126.6, 113.7, 86.4, 72.6, 64.7, 57.3, 56.9, 55.9, 43.4, 42.9, 41.2, 40.9, 37.2, 36.6, 36.3, 36.1, 35.3, 33.1, 31.3, 28.9, 27.9, 27.4, 27.2, 24.9, 24.1, 21.6, 19.4, 12.8; HRMS-FAB (m/z): [M-OH]⁺ calcd for C₄₅H₆₀O₄, 664.4492; found, 664.4489.

(3) Preparation of O-DMTr-((2-cyanoethyl)-N,N-diisopropyl-phosphoramidite)-lithocholic alcohol (Compound III)

O-DMTr-lithocholic alcohol (89.2 mg, 0.15 mmol) obtained in step 2 and DIPEA (77 μl, 0.45 mmol) were dissolved in 2 ml of CH₂Cl₂. Chloro-(2-cyanoethyl)-N,N-diisopropyl-phosphine (49 μl, 0.225 mmol) was added dropwise thereto and stirred for 15 min at room temperature. 10 ml of 5% NaHCO₃ was added thereto and the resulting solution was extracted with 10 ml of CH₂Cl₂. The organic layer was dried over MgSO₄ and evaporated under a reduced pressure. The resulting white solid residue was purified by flash chromatography (eluent —CH₂Cl₂:hexane=1:1.5) to obtain the title compound (69.3 mg, 0.081 mmol) as a white solid in a yield of 54%.

MS (FAB): m/z: 866 [M+H⁺]; IR (neat): v=3353, 2962, 2935, 2866, 1608, 1509, 1463, 1446, 1376, 1364, 1300, 1250, 1178, 1035, 975, 827, 754 cm⁻¹; ¹H-NMR (300 MHz, CDCl₃) δ=7.35 (d, 2H, J=7.6 Hz), 7.25-7.11 (m, 7H), 6.73 (d, 4H, J=8.7 Hz), 3.70 (s, 9H), 3.52 (m, 2H), 2.91 (m, 2H), 2.65 (t, 2H, J=6.4 Hz), 1.88-0.80 (m, 46H), 0.53 (s, 3H); ¹³C-NMR (75.5 MHz, CDCl₃) δ=159.0, 146.2, 137.6, 130.7, 129.0, 128.3, 127.2, 113.7, 110.1, 86.4, 77.9, 75.2, 74.9, 64.7, 59.1, 58.8, 57.2, 56.9, 55.9, 43.8, 43.7, 43.4, 43.0, 41.1, 40.9, 36.6, 36.2, 36.0, 35.3, 33.1, 32.3, 30.3, 28.9, 28.0, 27.4, 27.1, 25.4, 25.3, 25.2, 25.1, 24.9, 24.0, 21.5, 21.1, 21.0, 19.4, 12.7; ³¹P-NMR (121 MHz, CDCl₃) δ=148.1, 147.4; HRMS-FAB (m/z): [M+1]⁺ calcd for C₅₄H₇₈O₅N₂P₁, 865.5648; found, 865.5641.

### EXAMPLE 5

### Preparation of Phosphoramidite Compound of Formula (IVa) Used in the Synthesis of bDNA

(1) DMTr Protection Reaction of Pentaerithritol (Compound 10, 11, and 12)

Pentaerithritol (1.1 g, 7.34 mmol) and DMAP (4-dimethylaminopyridine, 276 mg, 2.26 mmol) were dissolved in 15 ml of Py/DMF (2/1), DMTr-Cl (4.1 g, 12.1 mmol) was added thereto and stirred for 10 hours at room temperature. 80 ml of 5% NaHCO₃ was added thereto and the resulting solution was extracted with 50 ml of CH₂Cl₂. The organic layer was dried over MgSO₄ and evaporated under a reduced pressure. The resulting orange colored oil residue was purified by flash chromatography (eluent—ethyl acetate:hexane=1:2 (compound 10); ethyl acetate:hexane=1:1 (compound 11); and CH₂Cl₂:MeOH=10:1 (compound 12)) to obtain the title compound (compound 10:2.93 g, 2.81 mmol, 70%; compound 11:520 mg, 0.702 mmol, 11.6%; and compound 12:395 mg, 0.901 mmol, 7.4%).

Compound 10: m.p. 96.3-97.8° C. MS (FAB): m/z: 1065.3 (M+Na⁺); IR (neat): v=3410.1, 2929.6, 1607.4, 1508.1, 1461.7, 1300.2, 1250.6, 1176.4, 1034.3 cm⁻¹; ¹H-NMR (300 MHz, CDCl₃): δ=7.26-7.24 (m, 6H), 7.19-7.15 (m, 21H), 6.72 (d, 12H, J=8.9 Hz), 3.76 (s, 18H), 3.59 (s, 2H), 3.32 (s, 2H); ¹³C-NMR (75.5 MHz, CDCl₃): δ=158.8, 145.3, 136.3, 130.6, 128.6, 128.1, 126.9, 113.4, 86.4, 64.3, 55.5, 45.8.

Compound 11: m.p. 88.8-89.7° C. MS (FAB): m/z: 763.2 (M+Na)⁺; IR (neat): v=3442, 1684, 1652, 1608, 1507, 1457, 1250, 1217, 1176, 1034 cm⁻¹, ¹H-NMR (300 MHz, CDCl₃): δ7.38-7.36 (m, 4H), 7.29-7.20 (m, 14H), 6.80 (4, 8H, J=8.5 Hz), 3.76 (s, 12H), 3.64 (s, 4H), 3.23 (s, 4H), 2.39 (s, 2H); ¹³C-NMR (75.5 MHz, CDCl₃): δ=158.0, 144.3, 135.3, 129.7, 127.7, 127.4, 126.3, 112.7, 85.8, 65.0, 62.7, 54.7, 45.0; HRMS-FAB (m/z): [M+Na]⁺ calcd for C₅₂H₅₄O₁₀Na, 763.3247; found, 763.3247.

Compound 12: an oil at room temperature; MS (FAB): m/z: 461.1 (M+Na⁺); IR (neat): v=3734.1, 3404.7, 2927.3, 1733.7, 1607.2, 1540.8, 1508.1, 1458.0, 1300.8, 1250.1, 1176.1, 1033.3, 828.9, 754.7 cm⁻¹, ¹H-NMR (300 MHz, CDCl₃): δ=7.40-7.39 (m, 2H), 7.31-7.24 (m, 7H), 6.84-6.81 (m, 4H), 3.77 (s, 6H), 3.71 (s, 6H), 3.16 (s, 2H), 2.35 (br, 2H), 1.63 (br, 1H); ¹³C-NMR (75.5 MHz, CDCl₃): δ=158.8, 135.7, 130.2, 128.2, 127.2, 113.5, 65.4, 64.1, 55.4, 45.5.

(2) Preparation of O-tri-DMTr-((2-cyanoethyl)-N,N-diisopropyl-phosphoramidite)-pentaerithritol (Compound IVa)

Compound 10 (560 mg, 0.537 mmol) obtained in step 1 and DIPEA (187 μl, 1.074 mmol) were dissolved in 6 ml of THF. Chloro-(2-cyanoethyl)-N,N-diisopropyl-phosphine (297 μl, 1.34 mmol) was added dropwise thereto and stirred for 1 hour at room temperature. 10 ml of 5% NaHCO₃ was added thereto and the resulting solution was extracted with 10 ml of ethyl acetate. The organic layer was dried over MgSO₄ and evaporated under a reduced pressure. The resulting yellow oil residue was purified by flash chromatography (eluent—ethyl acetate:hexane=1:3) to obtain the title compound (236 mg, 0.190 mmol) as a colorless oil in a yield of 35%.

MS (FAB): m/z: 1265.6 [M+Na⁺]; ¹H-NMR (300 MHz, CDCl₃): δ=7.27-7.14 (m, 27H), 6.72-6.68 (m, 12H), 4.11 (q, 2H, J=6.7 Hz), 3.75 (s, 18H), 3.39-3.23 (m, 8H), 2.23 (t, 2H, J=6.3 Hz), 2.03 (s, 2H), 1.31-1.22 (m, 4H), 1.09 (d, 6H, J=6.7 Hz), 0.95 (d, 6H, J=6.7 Hz); ¹³C-NMR (75.5 MHz, CDCl₃): δ=157.7, 144.7, 135.8, 129.7, 127.8, 127.1, 126.0, 112.5, 85.1, 62.3, 57.7, 54.7, 42.5, 24.1, 24.0, 13.7; ³¹P-NMR (121.5 MHz, CDCl₃): δ=148.9; HRMS-ESI (m/z): [M+Na]⁺ calcd for C₇₇H₈₃N₂O₁₁P₁Na₁, 1243.5852; found, 1243.5807.

### EXAMPLE 6

### Preparation of Phosphoramidite Compound of Formula (IVb) Used in the Synthesis of bDNA

(1) Preparation of O-Di-DMTr-O-Lev-pentaerithritol (Compound 13)

Compound 11 (506 mg, 0.68 mmol) obtained in step 1 of Example 5, EDC (288 mg, 1.50 mmol), and DMAP (184 mg, 1.50 mmol) were dissolved in 14 ml of CH₂Cl₂. Luvulinic acid (77 μl, 0.75 mmol) was added thereto and stirred for 3 hours at room temperature. 20 ml of 5% NaHCO₃ was added thereto and the resulting solution was extracted with 10 ml of CH₂Cl₂. The organic layer was dried over MgSO₄ and evaporated under a reduced pressure. The resulting residue was purified by flash chromatography (eluent—ethyl acetate:hexane=1:2) to obtain the title compound (319.7 mg, 0.37 mmol) as a yellow solid in a yield of 55%.

m.p. 55.4-56.1° C.; MS (FAB): m/z: 861.3 (M+Na⁺); IR (neat): v=3522.7, 3055.8, 3035.1, 3000.1, 2955.5, 2932.7, 2836.1, 1733.6, 1717.2, 1506.3, 1301.6, 1251.3, 1177.6, 1154.9, 1072.5, 1033.9 cm⁻¹; ¹H-NMR (300 MHz, Acetone-d₆): δ=7.41-7.38 (m, 4H), 7.29-7.18 (m, 14H), 6.85-6.82 (m, 8H), 4.15 (s, 2H), 3.77 (s, 12H), 3.77 (s, 12H), 3.69-3.67 (m, 2H), 3.50 (m, 1H), 3.29 (s, 4H), 2.63 (t, 2H, J=6.7 Hz), 2.35 (t, 2H, J=6.7 Hz), 2.07 (s, 3H); ¹³C-NMR (75.5 MHz, CDCl₃): δ=205.7, 172.4, 159.0, 145.9, 136.4, 130.6, 128.0, 126.9, 126.3, 113.3, 86.2, 64.0, 61.9, 55.0, 45.5, 37.7, 28.0; HRMS-FAB (m/z): [M+Na]⁺ calcd for C₅₂H₅₄O₁Na, 861.3615; found, 861.3617.

(2) Preparation of O-DMTr-O-di-Lev-O-((2-cyanoethyl)-N,N-diisopropyl-phosphoramidite)-pentaerithritol (Compound IVb)

O-Di-DMTr-O-Lev-pentaerithritol (319.7 mg, 0.37 mmol) obtained in step 1 and DIPEA (260 μl, 1.48 mmol) were dissolved in 4 ml of THF. Chloro-(2-cyanoetliyl)-N,N-diisopropyl-phosphine (166 μl, 0.74 mmol) was added dropwise thereto and stirred for 1.5 hours at room temperature. 10 ml of 5% NaHCO₃ was added thereto and the resulting solution was extracted with 10 ml of ethyl acetate. The organic layer was dried over MgSO₄ and evaporated under a reduced pressure. The resulting residue was purified by flash chromatography (eluent —CH₂Cl₂:hexane=3:2) to obtain the title compound (302.8 mg, 0.29 mmol) as a yellow oil in a yield of 77%.

MS (FAB): m/z: 1040 (M+H⁺); IR (neat): v=2964, 2931, 2932, 1736, 1720, 1607, 1581, 1508, 1463, 1444, 1250, 1177, 1032 cm⁻¹; ¹H-NMR (300 MHz, CDCl₃): δ=7.24 (d, 4H, J=7.2 Hz), 7.17-7.09 (m, 14H), 6.67 (d, 8H, J=8.4 Hz), 4.02 (q, 2H, J=10.7 Hz), 3.68 (s, 12H), 3.66-3.37 (m, 6H), 3.16 (m, 4H), 2.50 (t, 2H, J=7.0 Hz), 2.35 (t, 2H, J=6.3 Hz), 2.28 (t, 2H, J=6.7z), 2.05 (s, 3H), 1.21 (t, 4H, J=5.7 Hz), 1.05 (d, 6H, J=6.7 Hz), 0.94 (d, 6H, J=6.7 Hz); ¹³C-NMR (75,5 MHz, CDCl₃): δ=172.5, 158.8, 145.2, 136.2, 130.5, 128.4, 127.9, 126.8, 117.9, 113.2, 86.0, 61.6, 60.6, 55.4, 43.3, 43.1, 38.0, 30.0, 28.0, 24.8, 24.7, 14.4, 158.0, 144.2, 135.3, 135.0, 129.7, 127.7, 127.4, 127.3, 126.3, 117.2, 112.6, 112.5, 85.8, 85.5, 62.6, 61.6, 61.4, 60.2, 57.9, 57.7, 54.7, 46.9, 43.5, 42.7, 42.5, 37.3, 30.47, 29.3, 27.3, 24.2, 24.1, 22.1, 20.7, 19.9, 19.8; ³¹P-NMR (121.5 MHz, CDCl₃): 6=150.2; HRMS-FAB (m/z): [M+H]⁺ calcd for C₆₁H₇₂N₂O₁₁P₁Na₁, 1039.4874; found, 1039.4877.

### EXAMPLE 7

### Preparation of Phosphoramidite Compound of Formula (IVc) Used in the Synthesis of bDNA

(1) Preparations of O-DMTr-O-Lev-pentaerithritol (Compound 14) and O-DMTr-O-di-Lev-pentaerithritol (Compound 15)

Compound 12 (484.3 mg, 1.10 mmol) obtained in step 1 of Example 5, EDC (253 mg, 1.32 mmol) and DMAP (162 mg, 1.32 mmol) were dissolved in 10 ml of CH₂Cl₂. Luvulinic acid (135 mg, 1.32 mmol) was added thereto and stirred for 3 hours at room temperature. 20 ml of 5% NaHCO₃ was added thereto and the resulting solution was extracted with 15 ml of CH₂Cl₂. The organic layer was dried over MgSO₄ and evaporated under a reduced pressure. The resulting residue was purified by flash chromatography (eluent—ethyl acetate:hexane=1:2 (compound 14); and ethyl acetate:hexane=1:1 (compound 15)) to obtain the title compounds as yellow liquids (compound 14:141 mg, 0.22 mmol; and compound 15:285 mg, 0.53 mmol, 48%), respectively.

Compound 14: MS (FAB): m/z 657.2 (M+Na⁺); IR (neat): v=3390, 1792, 1772, 1699, 1684, 1653, 1558, 1540, 1521 cm ^{-1 1}H-NMR (300 MHz, CDCl₃): δ=7.41-7.38 (m, 2H), 7.28-7.20 (m, 7H), 6.85-6.81 (m, 4H), 4.16-4.10 (m, 4H), 3.78 (s, 6H), 3.54 (d, 2H, J=6.8 Hz), 3.14 (s, 2H), 2.69 (t, 4H, J=6.5 Hz), 2.50-2.44 (m, 4H), 2.15 (s, 6H), 1.25 (t, 1H, J=7.14 Hz); ¹³C-NMR-DEPT (75.5 MHz, CDCl₃): 5=130.7 (CH1), 128.7(CH₁), 128.5 (CH₁), 128.5 (CH₁), 113.8 (CH₁), 63.6 (CH₂), 62.6 (CH₂), 61.8 (CH₂), 55.9(CH₃), 38.6 (CH₂), 30.4 (CH₃), 28.5 (CH₂); HRMS-ESI (m/z): [M+Na]⁺ calcd for C₃₆H₄₂O₁₀Na, 657.2676; found, 657.2673.

Compound 15: MS (FAB): m/z 559.2 (M+Na⁺); IR (neat): v=3400, 3179, 3084, 3056, 3001, 2929.7, 2835.8, 1716.9, 1606.4, 1508.5, 1445.2, 1380.3, 1301.3, 1250.3, 1228.2, 1177.6, 1033.9, 996.5, 949.8, 830.4, 807.2, 754.7 cm⁻¹; ¹H-NMR (300 MHz, CDCl₃): δ=7.38-7.35 (m, 2H), 7.26-7.11 (m, 7H), 6.75 (d, 4H, J=8.5 Hz), 4.35 (s, 2H), 4.22(s, 2H), 3.68 (s, 6H), 3.64 (s, 4H), 3.08 (s, 2H), 2.57 (t, 2H, J=6.6 Hz), 2.37 (t, 2H, J=6.6 Hz), 2.05 (s, 3H); ¹³C-NMR (75.5 MHz, CDCl₃): δ=206.2, 172.4, 157.9, 153.8, 144.4, 135.4, 129.6, 127.6, 127.3, 126.2, 112.6, 85.5, 63.4, 63.2, 61.7, 54.6, 44.3, 29.3, 27.4; HRMS-FAB (m/z): [M+Na]⁺ calcd for C₃₁H₃₆O₈Na, 559.2308; found, 559.2308.

(2) Preparation of O-DMTr-O-di-Lev-O-((2-cyanoethyl)-N,N-diisopropyl-phosphoramidite)-pentaerithritol (Compound IVc)

O-DMTr-O-Lev-pentaerithritol (compound 14) (141 mg, 0.222 mmol) obtained in step 1 and DIPEA (77 μl, 0.445 mmol) were dissolved in 6 ml of THF. Chloro-(2-cyanoethyl)-N,N-diisopropyl-phosphine (74 μl, 0.33 mmol) was added dropwise thereto and stirred for 30 minutes at room temperature. 10 ml of 5% NaHCO₃ was added thereto and the resulting solution was extracted with 10 ml of ethyl acetate. The organic layer was dried over MgSO₄ and evaporated under a reduced pressure. The resulting residue was purified by flash chromatography (eluent—ethyl acetate:hexane=1: 3) to obtain the title compound (114.2 mg, 0.1354 mmol) as a yellow oil in a yield of 61%.

MS (FAB): IR (neat): v-2966, 2933, 1738, 1717, 1607, 1508, 1463, 1362, 1301, 1250, 1202, 1178, 1154, 1076, 1032 cm⁻¹; ¹H-NMR (300 MHz, CDCl₃): δ=7.40-7.38 (m, 2H), 7.29-7.26 (m, 7H), 6.82 (d, 4H, J=8.7 Hz), 4.17-4.12 (m, 4H), 3.78 (s, 6H), 3.73-3.65 (m, 2H), 3.61-3.51 (m, 2H), 3.15 (s, 2H), 2.68 (t, 4H, J=6.5 Hz), 2.56 (t, 2H, J=6.3 Hz), 2.48 (t, 4H, J=6.5 Hz), 2.16 (s, 6H), 1.25 (d, 2H, J=6.7 Hz), 1.16 (d, 6H, J=6.7 Hz), 1.10 (d, 6H, J=6.7 Hz); ¹³C-NMR (75,5 MHz, CDCl₃): δ=205.8, 171.8, 158.0, 158.0, 144.2, 135.3, 135.0, 129.7, 127.7, 127.4, 127.3, 126.3, 117.2, 112.6, 112.5, 85.8, 85.5, 62.6, 61.6, 61.4, 60.2, 57.9, 57.7, 54.7, 46.9, 43.5, 42.7, 42.5, 37.3, 30.47, 29.3, 27.3, 24.2, 24.1, 22.1, 20.7, 19.9, 19.8; ³¹P-NMR (121.5 MHz, CDCl₃): δ=150.7

### EXAMPLE 8

### Preparation of Phosphoramidite Compound of Formula (IVd) Used in the Synthesis of bDNA

(1) Preparation of O-DMTr-O-Lev-TBDMS-pentaerithritol (Compound 16)

O-DMTr-O-di-Lev-pentaerithritol (compound 15) (213 mg, 0.40 mmol) obtained in step 1 of Example 7 and DMAP (164 mg, 1.33 mmol) were dissolved in 4 ml of THF. Tert-Butyldimethylsilyl chloride (65 mg, 0.44 mmol) was added thereto and stirred for 3 hours at room temperature. 10 ml of 5% NaHCO₃ was added thereto and the resulting solution was extracted with 20 ml of CH₂Cl₂. The organic layer was dried over MgSO₄ and evaporated under a reduced pressure. The resulting residue was purified by flash chromatography (eluent—ethyl acetate:hexane=1:2) to obtain the title compound (119 mg, 0.18 mmol, 46%; Di-protected product: 72.8 mg, 0.1 mmol, 25%) as a yellow oil.

MS (FAB): m/z 673.3 M+Na⁺); IR (neat): v=3500.5, 2953.9, 2930.1, 2855.9, 1720.0, 1608.1, 1509.0, 1463.7, 1444.9, 1359.5, 1301.4, 1251.4, 1177.4, 1157.6, 1071.9, 1035.4, 911.9, 836.1 cm⁻¹; ¹H-NMR (300 MHz, CDCl₃): δ=7.43-7.41 (m, 2H), 7.32-7.26 (m, 7H), 6.85-6.82 (m, 4H), 4.20 (d, 2H, J=4.0 Hz), 3.79 (s, 6H), 3.66-3.64 (m, 4H), 3.15 (s, 2H), 2.70 (t, 2H, J=6.6 Hz), 2.50 (t, 2H, J=6.5 Hz), 2.17 (s, 3H), 0.84 (s, 9H), 0.03 (s, 3H), 0.02 (s, 3H); ¹³C-NMR (75.5 MHz, CDCl₃): δ=206.6, 173.0, 158.9, 145.1, 136.2, 130.5, 128.5, 128.2, 127.2, 113.5, 86.6, 64.8, 64.0, 63.9, 62.6, 55.6, 45.5, 38.3, 30.1, 28.3, 26.2, 18.5, −5.3; HRMS-FAB (m/z): [M+Na]⁺ calcd for, 673.3173; found, 673.3173.

(2) Preparation of O-DMTr-O-Lev-O-TBDMS-((2-cyanoethyl)-N,N-diisopropyl-phosphoramidite)-pentaerithritol (Compound IVd)

O-DMTr-O-Lev-TBDMS-pentaerithritol (compound 16) (134.8 mg, 0.207 mmol) obtained in step 1 and DIPEA (144 μl, 0.828 mmol) were dissolved in 4.2 ml of THF. Chloro-(2-cyanoethyl)-N,N-diisopropyl-phosphine (114 μl, 0.518 mmol) was added dropwise thereto and stirred for 1.5 hours at room temperature. 10 ml of 5% NaHCO₃ was added thereto and the resulting solution was extracted with 10 ml of ethyl acetate. The organic layer was dried over MgSO₄ and evaporated under a reduced pressure. The resulting residue was purified by flash chromatography (eluent—ethyl acetate:hexane=1:3) to obtain the title compound (86.2 mg, 0.101 mmol) as a yellow oil in a yield of 50%.

MS (FAB): m/z: 873.33 (M+Na⁺); IR(neat): v=2961.8, 2930.2, 2881.8, 2856.3, 1738.0, 1721.9, 1607.7, 1508.9, 1463.6, 1445.3, 1362.9, 1279.9, 1251.5, 1178.0 cm⁻¹; ¹H-NMR (300 MHz, CDCl₃): δ=7.41-7.38 (m, 2H), 7.29-7.17 (m, 7H), 6.79 (d, 4H, J=8.9 Hz), 4.11-4.09 (m, 2H), 3.76 (s, 6H), 3.70-3.52 (m, 8H), 3.12 (s, 2H), 2.66-2.64 (m, 2H), 2.53-2.45 (m, 4H), 2.15 (s, 3H), 1.14 (d, 6H, J=6.8 Hz), 1.08 (dd, 6H, J₁=6.7 Hz, J₂=1.4 Hz), 0.08 (d, 9H, J=1.1 Hz), −0.03 (d, 6H, J=2.4 Hz); ¹³C-NMR(75.5 MHz, CDCl₃): δ=205.9, 171.9, 157.9, 144.6, 135.7, 129.7, 127.2, 126.1, 117.2, 112.5, 85.3, 63.2, 61.0, 60.4, 57.7, 54.7, 45.0, 42.6, 42.5, 37.4, 29.4, 27.3, 25.3, 24.2, 24.1, 19.9, 19.8, 17.7, -6.12; ³¹P-NMR (121.5 MHz, CDCl₃): δ=150.3, 150.1; HRMS-FAB (m/z): [M+Na]⁺ calcd for C₄₆H₆₇N₂0₉P₁Si₁Na₁, 873.4251; found, 873.4252.

### EXAMPLE 9

### Preparation of Phosphoramidite Compound of Formula (V) Using Dendrimer

Dendrimer compound 17 (84 mg, 0.11 mmol, Hawker, C. J.; Frkchet, J. M. J. J. Am. Chem. SOC. 112, 7638-7647(1990)) and N-methyl morpholine (260 μl, 2.36 mmol) were dissolved in 4 ml of CH₃CN. Chloro-(2-cyanoethyl)-N,N-diisopropyl-phosphine (140 μl, 0.62 mmol) was added dropwise thereto and stirred for 5 minutes at room temperature. 10 ml of 5% NaHCO₃ was added thereto and the resulting solution was extracted with 15 ml of ethyl acetate. The organic layer was dried over MgSO₄ and evaporated under a reduced pressure. The resulting residue was purified by flash chromatography (eluent—ethyl acetate:hexane=1:3) to obtain the title compound (96 mg, 0.10 mmol) as a pale yellow oil in a yield of 92%.

¹H-NMR (300 MHz, CDCl₃): δ=7.32-7.23 (m, 20H), 6.61 (s, 4H), 6.50 (br, 4H), 6.47 (s, 1H), 4.94 (s, 8H), 4.88 (s, 4H), 4.49 (s, 2H), 4.11 (q, 2H), 3.92 (t, 2H), 2.28 (t, 2H), 1.04(d, 6H), 0.93 (d, 6H); ¹³C-NMR (75 MHz, CDCl₃): δ=160.9, 160.8, 140.0, 137.5, 129.3, 128.7, 128.3, 107.2, 106.5, 102.4, 70.9, 70.7, 53.0, 44.9, 44.8, 43.0, 22.5, 22.4; ³¹P-NMR (127 MHz, CDCl₃): δ=150.9.

While the invention has been described with respect to the above specific embodiments, it should be recognized that various modifications and changes may be made to the invention by those skilled in the art which also fall within the scope of the invention as defined by the appended claims.

What is claimed is:

1. A phosphoramidite compound of formula (I), (II, (III), (V) or (V): 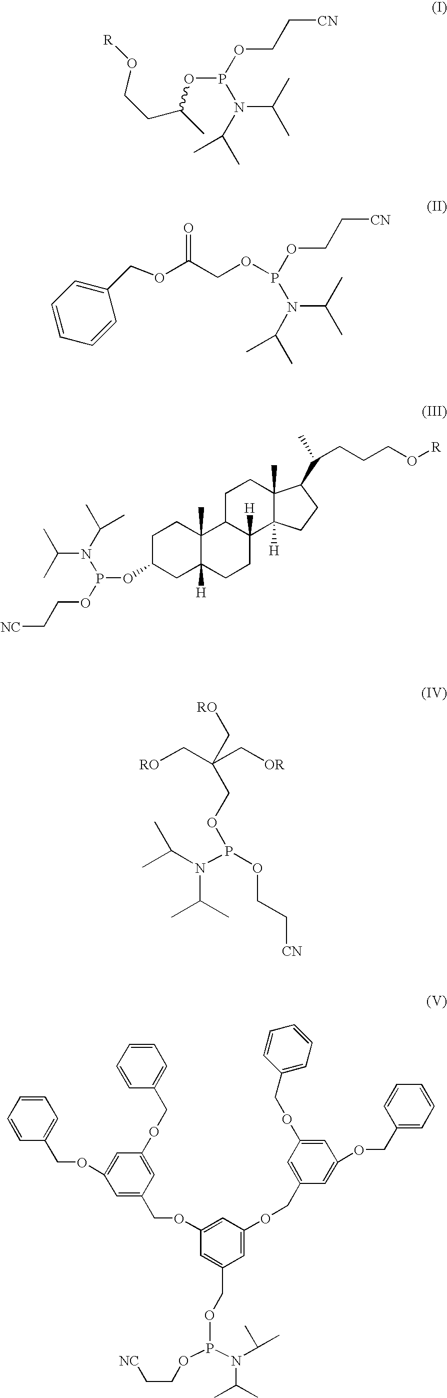
wherein:
R is a dimethoxytrityl(DMTr), levulinyl(Lev) or tert-butyldimethylsilyl (TBDMS) group.

2. The compound of claim 1, which is selected from the group consisting of:
(S)-(+)-1-O-DMTr-3-O-(2-cyanoethyl)-N,N-diisopropyl-phosphoramidite)-1,3-butanediol;
(R)-(−)-1-O-DMTr-3-O-(2-cyanoethyl)-N,N-diisopropyl-phosphoramidite)-1,3-butanediol;
O-((2-cyanoethyl)-N,N-diisopropyl-phosphoramidite)-benzylglycolate;
O-DMTr-((2-cyanoethyl)-N,N-diisopropyl-phosphoramidite)-lithocholic alcohol;
O-tri-DMTr-((2-cyanoethyl)-N,N-diisopropyl-phosphoramidite)-pentaerithritol;
O-DMTr-O-di-Lev-O-((2-cyanoethyl)-N,N-diisopropyl-phosphoramidite)-pentaerithritol; and
O-DMTr-O-Lev-O-TBDMS-((2-cyanoethyl)-N,N-diisopropyl-phosphoramidite)-pentaerithritol.


SEQUENCE LISTING (14 sequences)

SEQ ID NO: 1 - DNA, 12 bp, Artificial Sequence
  oligo 1
  modified_base 7: n is (R)-phosphoramidite compound
ttttttnttttt

SEQ ID NO: 2 - DNA, 12 bp, Artificial Sequence
  oligo 2
  modified_base 7: n is (S)-phosphoramidite compound
ttttttnttttt

SEQ ID NO: 3 - DNA, 12 bp, Artificial Sequence
  oligo 3
  modified_base 6: n is (R)-phosphoramidite compound
aaaaanaaaaaa

SEQ ID NO: 4 - DNA, 12 bp, Artificial Sequence
  oligo 4
  modified_base 6: n is (S)-phosphoramidite compound
aaaaanaaaaaa

SEQ ID NO: 5 - DNA, 12 bp, Artificial Sequence
  oligo 5
tttttttttttt

SEQ ID NO: 6 - DNA, 11 bp, Artificial Sequence
  oligo 6
ttttttttttt

SEQ ID NO: 7 - DNA, 12 bp, Artificial Sequence
  oligo 7
aaaaaaaaaaaa

SEQ ID NO: 8 - DNA, 11 bp, Artificial Sequence
  oligo 8
aaaaaaaaaaa

SEQ ID NO: 9 - DNA, 13 bp, Artificial Sequence
  oligo 9
  modified_base 7: n is (R)-phosphoramidite compound
aacgttnaacgtt

SEQ ID NO: 10 - DNA, 13 bp, Artificial Sequence
  oligo 10
  modified_base 7: n is (S)-phosphoramidite compound
aacgttnaacgtt

SEQ ID NO: 11 - DNA, 13 bp, Artificial Sequence
  oligo 11
aacgttaaacgtt

SEQ ID NO: 12 - DNA, 13 bp, Artificial Sequence
  oligo 12
aacgtttaacgtt

SEQ ID NO: 13 - DNA, 13 bp, Artificial Sequence
  oligo 13
aacgttgaacgtt

SEQ ID NO: 14 - DNA, 13 bp, Artificial Sequence
  oligo 14
aacgttcaacgtt